(19) United States
(12) Patent Application Publication
(10) Pub. No.: US 2019/0328991 A1
(43) Pub. Date: Oct. 31, 2019

(54) NASAL CANNULA FOR HIGH-FLOW VENTILATION

(71) Applicant: Drägerwerk AG & Co. KGaA, Lübeck (DE)

(72) Inventors: Helena KASZÁS, Groß Grönau (DE); Sebastian SCHRÖTER, Lübeck (DE); Katrin STURM, Lübeck (DE); Felix RIEDEL, Lübeck (DE)

(21) Appl. No.: 16/468,884

(22) PCT Filed: Dec. 6, 2017
(86) PCT No.: PCT/EP2017/081696

§ 371 Date: Jun. 12, 2019

(30) Foreign Application Priority Data
Dec. 13, 2016 (DE) 10 2016 014 751.2
May 3, 2017 (DE) 10 2017 004 224.1

(51) Int. Cl. A61M 16/06; A61M 16/08

(52) U.S. Cl. CPC A61M 16/0672; A61M 2206/11; A61M 16/0875

(57) ABSTRACT

A nasal cannula (1), for high-flow ventilation, includes a basic body (2) with a cavity (20). A tube connection element (6) provides a fluid-communicating connection of a tube (7) with the cavity (20). A first nasal prong and a second nasal prong (3, 4), respectively, each extend away from the basic body (20), for a fluid-communicating connection of each nostril each with the cavity (20). The first nasal prong (3) has a flow resistance different from that of the second nasal prong (4). The nasal cannula (1) can be simply manufactured and simply cleaned and avoids swirling of the breathing gas flow in the basic body (2) or clogging.

### CROSS REFERENCE TO RELATED APPLICATIONS

This application is a United States National Phase Application of International Application PCT/EP2017/081696, filed Dec. 6, 2017, and claims the benefit of priority under 35 U.S.C. § 119 of German Applications 10 2016 014 751.2, filed Dec. 13, 2016 and 10 2017 004 224.1 filed May 3, 2017, the entire contents of which are incorporated herein by reference.

### TECHNICAL FIELD

The present invention pertains to a nasal cannula for high-flow ventilation, wherein the nasal cannula has a basic body with a cavity, a tube connection element for the fluid-communicating connection of a tube with the cavity, and a first and second nasal prongs pointing away from the basic body for the fluid-communicating connection of each nostril with the cavity.

### BACKGROUND

Patients who can breathe on their own but additionally need oxygen are treated with a high-flow oxygen therapy. Heated and humidified breathing gas in the form of a mixture of oxygen and air is supplied to the patient in this therapy. This improves the oxygenation of the patient. A nasal cannula is used as the interface between the ventilator and the patient. There are variants in this connection in which each nostril is treated with a separate tube and in which only one tube is used for both nostrils to keep the number of tube connections low. The present invention pertains to the alternative with one tube connection for both nostrils.

In case of a nasal cannula with one tube connection, the tube is led from one side of the patient to the nasal cannula. The nasal cannula has two hollow nasal prongs here, which are inserted into one of the nostrils each. One of the nasal prongs is arranged closer to the tube than the other. As a result, a dynamic pressure develops at the inlet of the nasal prong facing away from the tube, this pressure developing due to the deflection of the breathing gas flow at the inlet of this nasal prong. As a result, more gas flows through the tube-side nasal prong than through the nasal prong that is arranged on the side facing away from the tube. This leads to a nonuniform incoming flow into the two nostrils of the patient. This may be unpleasant for the patient.

A nasal cannula with a hollow basic body and with only one tube connection from one side is described according to WO 2015/041546 A1. The basic body has a restriction between the nasal prongs in order to distribute the breathing gas flow uniformly between the two nasal prongs. The drawback of this device is that it is difficult to manufacture and to clean it. Further, swirling, which may again abolish the uniform distribution of the breathing gas flow, may develop in the basic body. Furthermore, a part of the restriction may break off and clog the basic body in case of material defects.

### SUMMARY OF THE INVENTION

An object of the present invention is therefore to provide a nasal cannula, which can be manufactured and cleaned in a simple manner and avoids swirling of the breathing gas flow in the basic body or clogging.

Provisions are made according to the present invention in a nasal cannula for high-flow ventilation, wherein the nasal cannula has a basic body with a cavity, a tube connection element for the fluid-communicating connection of a tube with the cavity, and a first nasal prong and a second nasal prong pointing away from the basic body for the fluid-communicating connection of one nostril each with the cavity, for the first nasal prong to have a flow resistance different from that of the second nasal prong.

A basic concept of the present invention is to regulate the flow of breathing gas in the two nasal prongs by means of the different flow resistances. A homogeneous pressure distribution is brought about in the cavity by the two different flow resistances in the prongs. An essentially equal breathing gas flow flows through the two nasal prongs due to the homogeneous pressure distribution.

It was recognized in the present invention that the combination of the flow resistance in the cavity arranged upstream between the respective nasal prong and the tube connection element determines the flow of breathing gas through the respective nasal prong. The overall flow resistances on the paths between the tube connection element and the first and second nasal prongs are equalized with one another by the different flow resistances of the first and second nasal prongs. A breathing gas flow that is essentially equal develops as a result in the two nasal prongs.

Further, the nasal cannula according to the present invention can be cleaned easily, because the nasal prongs are readily accessible from the outside. Clogging of the basic body by broken-off parts is also avoided, because the breathing gas flow is directed out of the basic body and thus it removes small broken-off parts from the nasal cannula. Furthermore, swirling is avoided in the basic body by this arrangement.

The first nasal prong is advantageously arranged closer to the tube connection element than the second nasal prong, and the first nasal prong has a lower flow resistance than the second nasal prong. The present invention is thus based on the special discovery that it is advantageous for achieving a uniform flow through the nasal prongs if the first nasal prong has a lower resistance than the second one, even though the first one is arranged closer to the tube connection element than the second nasal prong, so that the flow path from the oxygen supply unit to the outlet of the respective nasal prong is shorter during the flow through the first nasal prong than during the flow through the second nasal prong.

Further, it is advantageous that an internal diameter averaged along the longitudinal axis of the first nasal prong is different from an internal diameter averaged along the longitudinal axis of the second nasal prong. The longitudinal axis of a nasal prong is defined here as the axis along the cavity of the nasal prong. The walls of the nasal prong extend around the longitudinal axis.

The internal diameter averaged along the longitudinal axis of the first nasal prong is advantageously larger than the internal diameter averaged along the longitudinal axis of the second nasal prong. The second nasal prong advantageously has an element for increasing the flow resistance. Since the first nasal prong is arranged closer to the tube connection element than the second nasal prong, the dynamic pressure area, which develops in the cavity at the second nasal prong, is increased over the entire cavity up to the first nasal prong. As a result, a homogeneous pressure is generated in the cavity, so that the breathing gas flows through the first and second nasal prongs are equalized. It was surprisingly found in this connection that at equal dimensions of the first and second nasal prongs, the larger percentage of the flow flows through the second nasal prong, even though the flow path is longer during the flow through the nasal cannula. Using the present invention, the flow resistance is therefore advantageously increased in the flow path through the first nasal prong in order to make the volume flows flowing through the two nasal prongs, on the whole, uniform or to adapt them to one another.

In an alternative embodiment to this, the internal diameter of the first nasal prong is advantageously larger than the internal diameter of the second nasal prong. The second nasal prong advantageously has a restriction element here.

It is also advantageous that an outlet opening of the first nasal prong has the same diameter as an outlet opening of the second nasal prong. Identical components can thus be used when manufacturing the nasal prongs, as a result of which the manufacturing costs decrease. Further, a subjective impression of the patient that different air flows flow through the two nasal prongs because of outlet openings having different sizes is avoided.

The outlet openings may advantageously have a diameter between 1 mm and 15 mm, preferably between 2 mm and 10 mm and more preferably between 3.5 mm and 6 mm.

Further, the second nasal prong may advantageously have, starting from the cavity, first a conically tapering section and, adjoining this, a cylindrically shaped section, and the first nasal prong has a cylindrically shaped section starting from the cavity. The flow resistance of the nasal prong is increased by the conically tapered section, because the density of the fluid flowing through the tapering section increases due to the tapering.

It is advantageous in this connection if the conically tapering section has an opening angle between 2° and 6°, preferably between 3° and 5° and more preferably 4°.

Further, the second nasal prong advantageously has a surface on an inner wall area that has a higher flow resistance than a surface in an inner wall area of the first nasal prong.

Further, the conically tapering section preferably has a length of 1 mm to 6 mm, preferably 3.5 mm, the cylindrically shaped section having a length of 5 mm to 10 mm and preferably 7.3 mm.

The present invention will be explained in more detail below on the basis of an advantageous exemplary embodiment by means of the attached drawings. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a perspective schematic view showing a of a nasal cannula;

FIG. 2a is a schematic sectional view of the nasal cannula with one flow resistance in the nasal prongs;

FIG. 2b is a schematic sectional view of the nasal cannula with another, different flow resistance in the nasal prongs;

FIG. 2c is a schematic sectional view of the nasal cannula with another, different flow resistance in the nasal prongs;

FIG. 2d is a schematic sectional view of the nasal cannula with another, different flow resistance in the nasal prongs; and

FIG. 2e is a schematic sectional view of the nasal cannula with another, different flow resistance in the nasal prongs.

### DESCRIPTION OF PREFERRED EMBODIMENTS

Referring to the drawings, a nasal cannula for high-flow ventilation is generally designated 1.

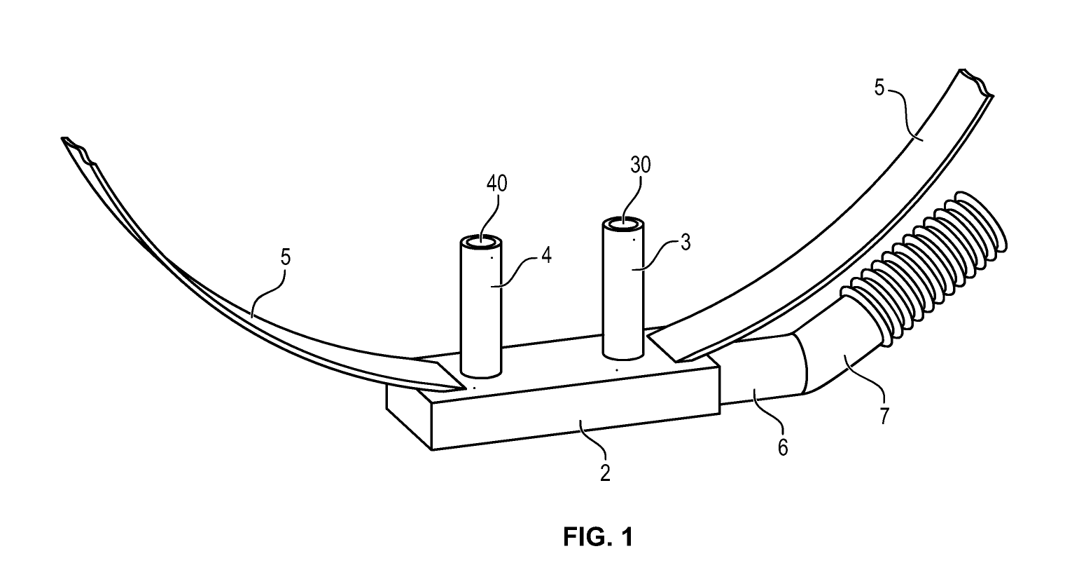

The nasal cannula 1 comprises, according to FIG. 1, a basic body 2, from which a first nasal prong 3 and a second nasal prong 4 branch off. The basic body 2 further comprises a tube connection element 6, which can be connected to a ventilation tube 7. The nasal cannula 1 further comprises a holding element 5, with which the nasal cannula can be attached to a head band and can thus be placed on the face of a patient.

The basic body 2 further has a cavity 20, which is connected to the first nasal prong 3 and to the second nasal prong 4 in a fluid-communicating manner. The nasal prongs 3, 4 form a connection of the cavity with the surrounding area. When the nasal prongs 3, 4 are inserted into the nostrils, the cavity 20 is connected to the nostrils via the nasal prongs 3, 4 in a fluid-communicating manner.

The cavity 20 further has a fluid-communicating connection with the tube connection element 6. A breathing gas flow, which flows into the nasal cannula 1 through the tube connection element 6, is now distributed via the cavity 20 between the nasal prongs 3, 4.

The tube connection element 6 is arranged laterally relative to the orientation of the nasal prongs 3, 4. This means that an air flow, which flows into the cavity 20 through the tube connection element 6, must make a change in direction within the cavity 20 in order to flow through the nasal prongs 3, 4.

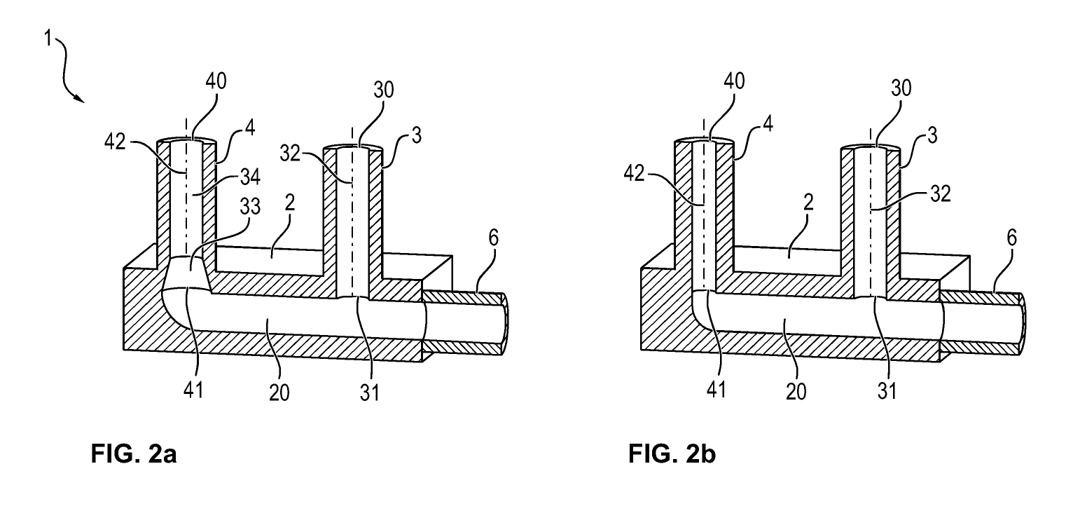

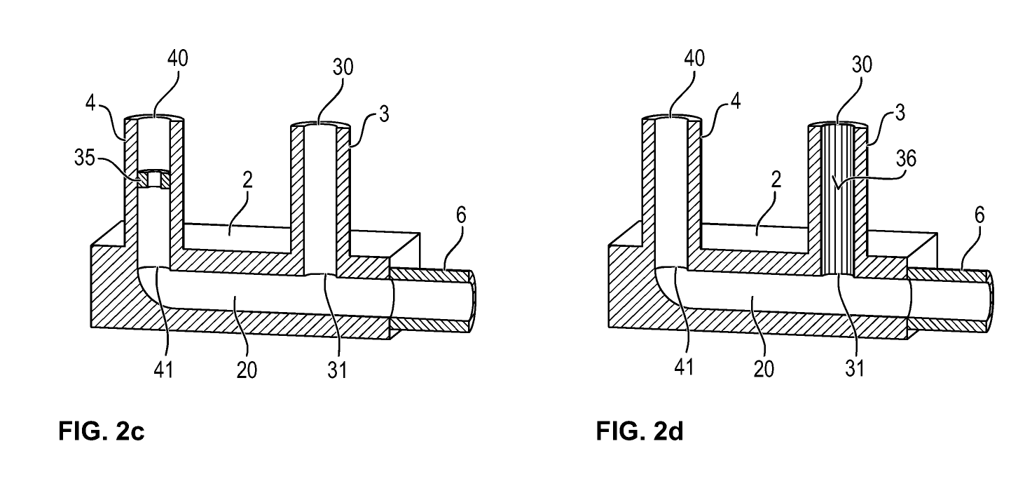

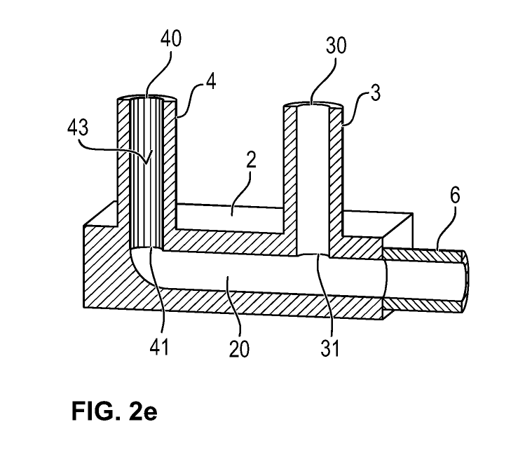

FIG. 2a shows a schematic sectional view through a first embodiment of a nasal cannula 1. The cavity 20 within the basic body 2 is connected here via a first inlet opening 31 to the first nasal prong 3 in a fluid-communicating manner. The cavity 20 is connected to the second nasal prong 4 via the second inlet opening 41 in a fluid-communicating manner.

The first nasal prong 3 is arranged closer to the tube connection element 6 than the second nasal prong 4. The second nasal prong 4 has, starting from the second inlet opening 41, a conically tapering section 33, which is adjoined by a cylindrical section 42, which then opens into a second outlet opening 40 of the second nasal prong 4.

The first outlet opening 30 and the second outlet opening 40 have the same diameter. The result is that the second inlet opening 41 is larger than the first opening 31. As a result, the internal diameter of the second nasal prong 4, averaged along the longitudinal axis 42, is larger than the internal diameter along a first longitudinal axis 32 of the first nasal prong 3.

The conically tapering section 33 now causes the breathing gas flow, which flows through the second nasal prong 4, to be dammed up, so that the flow resistance increases in the second nasal prong 4. This brings about a backup into the cavity 20, so that a smaller breathing gas flow flows through the second nasal prong 4 compared to a nasal cannula 1 that is not configured according to the present invention.

The diameters of the outlet openings 30, 40 are between 1 mm and 15 mm, preferably between 2 mm and 10 mm, and especially preferably between 3.5 mm and 6 mm. Further, the conically tapering section has an opening angle between 2° and 6°, preferably 3° to 5° and especially preferably 4°. The conically tapering section has a length of 1 mm to 6 mm and preferably 3.5 mm, the cylindrically shaped section of the first nasal prong having a length of 5 mm to 10 mm and preferably 7.3 mm.

The tube connection element 6 may be configured as a male or female connection piece. A ventilation tube 7 may thus either be in contact with the tube connection element 6 on the outside or the ventilation tube 7 is inserted into the tube connection element 6.

FIG. 2b shows a second embodiment of the present invention. The nasal prongs 3, 4 have different flow resistances here, which are brought about by different internal diameters of the nasal prongs 3, 4. The first nasal prong 3, which is arranged closer to the tube connection element, has a larger internal diameter than does the second nasal prong 4, which is arranged at a greater distance from the tube connection element 6. The internal diameter of a nasal prong 3, 4 is constant over the entire length of the respective nasal prong 3, 4.

The thickness of the tube wall of the nasal prong 3, 4 may be selected to be variable corresponding to the prong diameter, so that both nasal prongs 3, 4 have equal external diameters.

The larger internal diameter of the first nasal prong 3 compared to the smaller internal diameter of the second nasal prong 4 causes the flow resistance to be higher in the second nasal prong 4 than in the first nasal prong 3. The breathing gas flow through the first nasal prong 3 is therefore increased compared to the second nasal prong 4. Since less breathing gas flows through the first nasal prong 3 at equal flow resistances in the first nasal prong 3 and in the second nasal prong 4, a balanced ratio of the breathing gas flow through the first and second nasal prongs 3, 4 is brought about by the larger internal diameter selected for the first nasal prong 3 compared to the internal diameter of the second nasal prong 4.

A third embodiment is shown in FIG. 2c. The first nasal prong 3 and the second nasal prong 4 have the same internal diameter here along the respective longitudinal axes 32, 42, i.e., the inlet openings 31, 41 and the outlet openings 30, 40 have the same diameter.

Contrary to the first nasal prong 3, the second nasal prong 4 has a flow path restriction element 35. The restriction element 35 is configured in this embodiment as an insert into the second nasal prong 4. This insert has an external diameter that corresponds to the internal diameter of the nasal prong 4. Further, the insert comprises a hole, which has a smaller diameter than the internal diameter of the nasal prong 4. As a result, a narrowing is created in the flow path of the second nasal prong 4.

The flow resistance of the second nasal prong 4 is increased by the restriction element 35. As a result, the first nasal prong 3 has a lower flow resistance than the second nasal prong 4. The air flows through the nasal prongs 3, 4 can be equalized with one another in this manner by means of the restriction element 35.

FIG. 2d shows a fourth embodiment of the nasal cannula 1. The first nasal prong 3 and the second nasal prong 4 have the same internal diameter here along the longitudinal axis. However, the first nasal prong 3 has a surface element 36, which has a coating and/or surface structure, which reduces the friction between the air flow and the inner wall of the first nasal prong 3.

The surface element 36 has a grooved structure, which reduces the flow resistance of the breathing gas flow, which flows through the first nasal prong 3.

A coating of the surface element 36 has a material that has a lower coefficient of friction with the incoming air flow.

A lower flow resistance can be brought about in this manner in the first nasal prong 3 compared to the second nasal prong 4.

In a fifth embodiment according to FIG. 2e, the second nasal prong 4 has a surface element 43 with a coating and/or surface structure that increases the flow resistance of the second nasal prong 4.

The surface structure of the surface element 43 may have projections, which generate turbulences in the breathing gas flow through the second nasal prong 4. The flow resistance of the second nasal prong 4 is increased by the turbulences in the breathing gas flow of the second nasal prong 4.

A coating of the surface element 36 of the second nasal prong 4 may have a high coefficient of friction with the incoming air flow. The edges of the breathing gas flow, which flows through the second nasal prong 4, are thus subjected to a high friction compared to the inner wall of the first nasal prong 3. This increases the flow resistance of the breathing gas flow in the second nasal prong 4.

A flow resistance that is different from that in the first nasal prong 3 is brought about in this manner in the second nasal prong 4 in this embodiment as well by means of the surface element 36, which increases the flow resistance of the second nasal prong 4.

A lower flow resistance can be brought about in this manner in the first nasal prong 3 compared to the second nasal prong 4.

The above-described embodiments of the nasal cannula 1 may be combined with one another as needed, so that the nasal cannula 1 has, for example, a surface element 43 with a coating with a high coefficient of friction in the second nasal prong 4 and a surface element 36 with a surface structure that has a low coefficient of friction in the first nasal prong 3. The other embodiments described may also be combined with one another in a similar manner.

While specific embodiments of the invention have been shown and described in detail to illustrate the application of the principles of the invention, it will be understood that the invention may be embodied otherwise without departing from such principles.

1. A nasal cannula for high-flow ventilation, the nasal cannula comprising:
a basic body with a cavity
a tube connection element for the fluid-communicating connection of a tube with the cavity;
a first nasal prong; and
a second nasal prong, the first nasal prong and the second nasal prong, respectively, extending away from the basic body, to each provide a fluid-communicating connection of a nostril with the cavity, wherein the first nasal prong has a flow resistance different from that of the second nasal prongs, the first nasal prong is arranged closer to the tube connection element than the second nasal prong and the first nasal prong has a lower flow resistance than the second nasal prong.

2. A nasal cannula in accordance with claim 1, wherein a first nasal prong internal diameter, averaged along a longitudinal axis of the first nasal prong is different from a second nasal prong internal diameter, averaged along a longitudinal axis of the second nasal prong.

3. A nasal cannula in accordance with claim 2, wherein the first nasal prong internal diameter averaged along the longitudinal axis of the first nasal prong is larger than the second nasal prong internal diameter averaged along the longitudinal axis of the second nasal prong.

4. A nasal cannula in accordance with claim 1, wherein the second nasal prong has a flow path restriction.

5. A nasal cannula in accordance with claim 1, wherein an outlet opening of the first nasal prong has the same diameter as an outlet opening of the second nasal prong.

6. A nasal cannula in accordance with claim 5, wherein outlet openings of the first nasal prong and the second nasal prong have a diameter between 1 mm and 15 mm.

7. A nasal cannula in accordance with claim 1, wherein:
the second nasal prong has, starling from the cavity, first a conically tapering section followed by a cylindrically shaped section: and
the first nasal prong has, starting from the cavity, a cylindrically shaped section.

8. A nasal cannula in accordance with claim 7, wherein the conically tapering section has an opening angle between 2° and

9. A nasal cannula in accordance with claim 7, wherein the conically tapering section has a length of 1 mm to 6 mm and the cylindrically shaped section has a length of 5 mm to 10 mm.

10. A nasal cannula in accordance with claim 2, wherein an internal diameter of the first nasal prong is larger than the internal diameter of the second nasal prong.

11. A nasal cannula in accordance with claim 2, wherein the second nasal prong has a surface on an inner wall area that has a higher flow resistance than a surface in an inner wall area of the first nasal prong.

12. A ventilation nasal cannula comprising:
a basic body defining a flow cavity;
a tube connection element defining a connection element flow passage, the tube connection element being connected to the basic body with the connection element flow passage in fluid-communicating connection with the flow cavity;
a first nasal prong defining a first nasal prong flow passage, the first nasal prong flow passage being in fluid-communicating connection with the flow cavity and having a first nasal prong flow passage flow resistance; and
a second nasal prong defining a second nasal prong flow passage, the second nasal prong flow passage being in fluid-communicating connection with the flow cavity and having a second nasal prong flow passage flow resistance, wherein the first nasal prong is arranged closer to the tube connection element than the second nasal prong and the first nasal prong flow passage flow resistance is lower than the second nasal prong flow passage flow resistance.

13. A nasal cannula in accordance with claim 12, wherein:
the first nasal prong flow passage has a first passage internal diameter, averaged along a longitudinal axis of the first nasal prong;
the second nasal prong flow passage has a second passage internal diameter, averaged along a longitudinal axis of the second nasal prong;
the first passage internal diameter is larger than the second passage internal diameter.

14. A nasal cannula in accordance with claim 13, wherein the second nasal prong has a flow path restriction element.

15. A nasal cannula in accordance with claim 12, wherein:
the first nasal prong flow passage has a first passage outlet opening;
the second nasal prong flow passage has a second passage outlet opening;
the first passage outlet opening has a diameter that is essentially the same as a diameter of the second passage outlet opening.

16. A nasal cannula in accordance with claim 15, wherein the diameter of the first passage outlet opening and the second passage outlet opening is from 1 mm to 15 mm.

17. A nasal cannula in accordance with claim 12, wherein:
the first nasal prong flow passage, starting from the cavity, comprise a conically tapering section followed by a cylindrically shaped section; and
the second nasal prong flow passage, starting from the cavity, comprises a cylindrically shaped section.

18. A nasal cannula in accordance with claim 17, wherein the conically tapering section has an opening angle between 2° and 6°.

19. A nasal cannula in accordance with claim 17, wherein the conically tapering section has a length of 1 mm to 6 mm.

20. A nasal cannula in accordance with claim 12, wherein:
the first nasal prong flow passage is delimited by an inner wall surface having a first inner wall resistance;
the second nasal prong flow passage is delimited by an inner wall surface having a second wall resistance;
the second wall resistance is higher than the first inner wall resistance.
